(19) United States
(12) Patent Application Publication
(10) Pub. No.: US 2011/0196372 A1
(43) Pub. Date: Aug. 11, 2011

(54) Bone Fixing Material and Thighbone Fixing System

(75) Inventor: Takahiro Murase, Bunkyo-Ku (JP)

(73) Assignee: ROBERT REID, INC., Bunkyo-ku (JP)

(21) Appl. No.: 13/125,483

(22) PCT Filed: Mar. 30, 2009
(86) PCT No.: PCT/JP2009/056549

§ 371 Date: Apr. 21, 2011

(30) Foreign Application Priority Data
Oct. 31, 2008 (JP) 2008-281790

(51) Int. Cl. A61B 17/58

(52) U.S. Cl. 606/64

(57) ABSTRACT

A bone fixing blade (2), which is fixed at a intermedullary nail main body (1) to be inserted into a medullary space of a fractured thighbone and which fixes a big bone piece portion (B1) and a small bone piece portion (B2) composing the thighbone, is provided with a cut-out portion forming unit (231) formed in a stick-like shape along a circumferential direction of the bone fixing blade comprised of three cut-out portions (23a) cut out from the surface toward the inside, wherein the cut-out portion forming unit (231) is pressed into the inside of each of the big bone piece portion and the small bone piece portion to be in a position on a recess side that is deeper than a bone fracture line (L).

### TECHNICAL FIELD

The present invention relates to a bone fixing material and a thighbone fixing system to fix a fractured thighbone.

### BACKGROUND ART

Conventionally, there is known a thighbone fixing system to treat a fracture of a proximal end of the thighbone such as a thighbone neck portion (for example, see Patent Documents 1 and 2).

Specifically, the thighbone fixing system includes an intramedullary nail main body to be inserted to a medullary space of a thighbone from a proximal end of the thighbone; a bone fixing material to be inserted through an inserting hole formed so as to cross with a longitudinal direction of the intramedullary nail main body and the bone fixing material is positioned so that a tip section is positioned in the bone head of the thighbone; and a fixing screw to fix the bone fixing material inserted through the inserting hole so as not to rotate.

As a bone fixing material, for example, there is known a screw type which is screwed into the bone head and a blade type which is pressed into the bone head.

Regarding these material, as for the screw type bone fixing material, considering screwing the material in the thighbone, it is preferable that the tip section is formed in a more pointed shape. However, if the tip section is too pointed, there is a possibility that the tip section pierces through the outside of the bone head after surgery. Therefore, the bone fixing material is formed in a cylinder shape and after forming a pilot hole on the inside of the bone head from the side of the thighbone with a reamer, the bone fixing material is screwed in.
Patent Document 1: Japanese Patent Application Laid-Open Publication No. 2008-36094
Patent Document 2: Japanese Patent Application Laid-Open Publication No. 2007-61631

### DISCLOSURE OF THE INVENTION

### Problems To Be Solved By the Invention

However, since the cancellous bone mass decreases due to forming the pilot hole, there is a possibility that a problem occurs, such as the strength of the threaded engagement of the bone fixing material is weak even if the bone fixing material is screwed in. Specifically, in those with small cancellous bone mass such as those who have weak bones or those who are aged, there is a possibility that the strength of the threaded engagement of the bone fixing material becomes weak. Therefore, the bone fixing material easily falls out and in a case of fracture of the thighbone neck bone, the bone piece portion on the bone head side easily rotates.

Alternatively, as a blade type bone fixing material, there is known a material formed so that the cross section substantially orthogonal to the longitudinal direction is in a curved shape so that free end sections of each of the top end and the bottom end of an H shape are close to each other. With such bone fixing material, the amount of the cancellous bone removed when the bone fixing material is provided in the thighbone is small compared to the screw type bone fixing material. Therefore, the speed of bone adhesion is faster and there is an advantage that even in a fracture of the thighbone neck portion, the bone piece portion on the bone head side does not easily rotate.

However, since the blade type bone fixing material is supported by pressure from the surrounding cancellous bone, when the cancellous bone mass is small, there is a problem that the bone fixing material falls out easily as in the screw type bone fixing material.

Therefore, the object of the present invention is to provide a bone fixing material and a thighbone fixing system in which the material falling out from the thighbone can be appropriately prevented.

### Means for Solving the Problem

According to an embodiment of the present invention, there is provided a bone fixing material attached and fixed to an intramedullary nail main body inserted in a medullary cavity of a fractured thighbone to fix one bone piece portion and the other bone piece portion different from the one bone piece portion composing the thighbone, the bone fixing material including:

a form formed in a stick shape; and

a cut out section forming section provided with at least one cut out section formed along a direction which crosses with a longitudinal direction of the bone fixing material and cut out from a surface to an inner side, wherein

the bone fixing material is pressed into an inner side of each of the one bone piece portion and the other bone piece portion so that the cut out section forming section is positioned in a deeper side than the bone fracture line.

Moreover, the bone fixing material may further include at least one groove section formed along the longitudinal direction at a tip section side pressed in first into the one bone piece portion and the other bone piece portion, the groove section formed curved to the inner side from the surface.

Moreover, in the bone fixing material, it is possible that the cut out section forming section and the groove section are each provided in a plurality of units and are provided alternately in a circular direction substantially orthogonal to the longitudinal direction of the bone fixing material.

Moreover, in the bone fixing material, it is possible that:

the cut out section forming section is provided in a plurality of units with a predetermined space in between each unit in the circular direction substantially orthogonal to the longitudinal direction; and

the cut out sections provided in each of the plurality of cut out section forming sections and adjacent to each other in the circular direction are provided misaligned in the longitudinal direction.

Moreover, in the bone fixing material, it is possible that the cut out section is cut out deeper toward a tip section side pressed in first into the one bone piece portion and the other bone piece portion.

Moreover, it is possible that a thighbone fixing system includes:

the bone fixing material; and

an intramedullary nail main body formed in a stick shape, wherein

the intramedullary nail main body includes an inserting hole forming section to form an inserting hole to insert the bone fixing material; and

a width of the inserting hole forming section in a direction substantially orthogonal to a longitudinal direction of the intramedullary nail main body and in a direction which does not cross with the inserting direction of the bone fixing material is larger than a width in a direction which crosses with the inserting direction.

### Advantageous Effect of the Invention

According to the present invention, the bone fixing material attached and fixed to the intramedullary nail main body includes a form formed in a stick shape; and a cut out section forming section provided with at least one cut out section formed along a direction which crosses with a longitudinal direction of the bone fixing material and cut out from a surface to an inner side, wherein the bone fixing material is pressed into an inner side of each of the one bone piece portion and the other bone piece portion composing the thighbone so that the cut out section forming section is positioned in a deeper side than the bone fracture line. Therefore, the cancellous bone surrounding the bone fixing material is in a state in the inner side of the cut out section after surgery, and even if the bone fixing material is moved in the axis direction, the cut out section is easily caught by the cancellous bone. Consequently, the bone fixing material can be made so that it does not easily fall out from the thighbone. Moreover, with the cut out section, the surface area of the bone fixing material can be made larger, and therefore can be appropriately supported by the pressure from the cancellous bone surrounding the bone fixing material in a state pressed in the inner side of the one bone piece portion and the other bone piece portion.

Therefore, the bone fixing material falling out from the thighbone can be appropriately prevented.

### BRIEF DESCRIPTION OF THE DRAWINGS

FIG. 1 is a diagram schematically showing a thighbone fixing system illustrated as a preferable embodiment employing the present invention, the thighbone fixing system illustrated from a front side;

FIG. 2 is an exploded perspective diagram showing a thighbone fixing system of FIG. 1;

FIG. 3A is a diagram showing an intramedullary nail main body composing the thighbone fixing system of FIG. 1 viewed from the front side;

FIG. 3B is a diagram showing the intramedullary nail main body of FIG. 3A viewed from the right side;

FIG. 3C is a diagram showing the intramedullary nail main body of FIG. 3A viewed from the upper side;

FIG. 4 is a perspective diagram showing a bone fixing blade composing the thighbone fixing system of FIG. 1;

FIG. 5A is a diagram showing the bone fixing blade of FIG. 4 viewed from the front side;

FIG. 5B is a diagram showing the bone fixing blade of FIG. 5A viewed from the rear side;

FIG. 6 is a perspective view showing a bone fixing screw composing the thighbone fixing system of FIG. 1; and

FIG. 7 is a diagram showing a modified example of a bone fixing blade from a side view.

### BEST MODE FOR CARRYING OUT THE INVENTION

Below, a specific embodiment of the present invention is described with reference to the drawings. However, the scope of the invention is not limited to the illustrated examples.

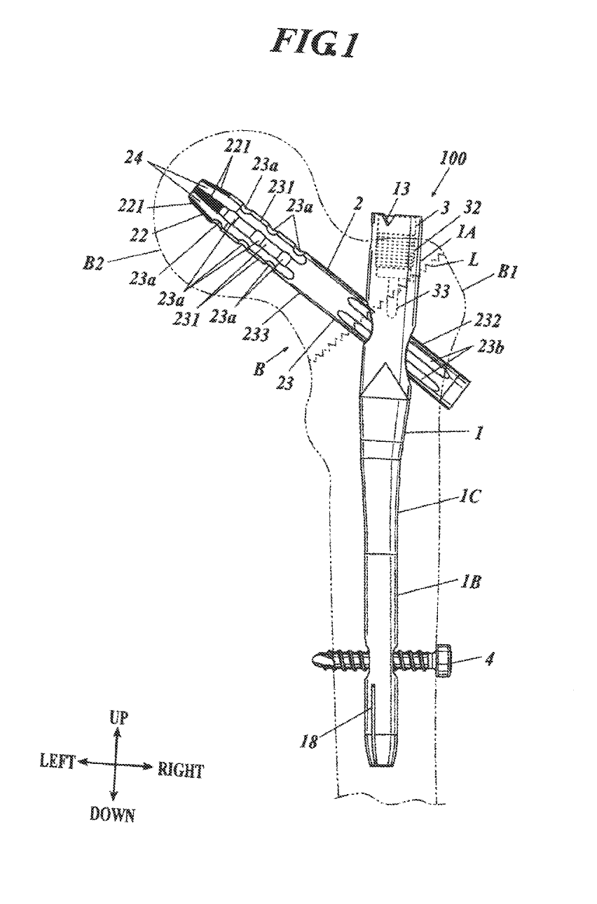

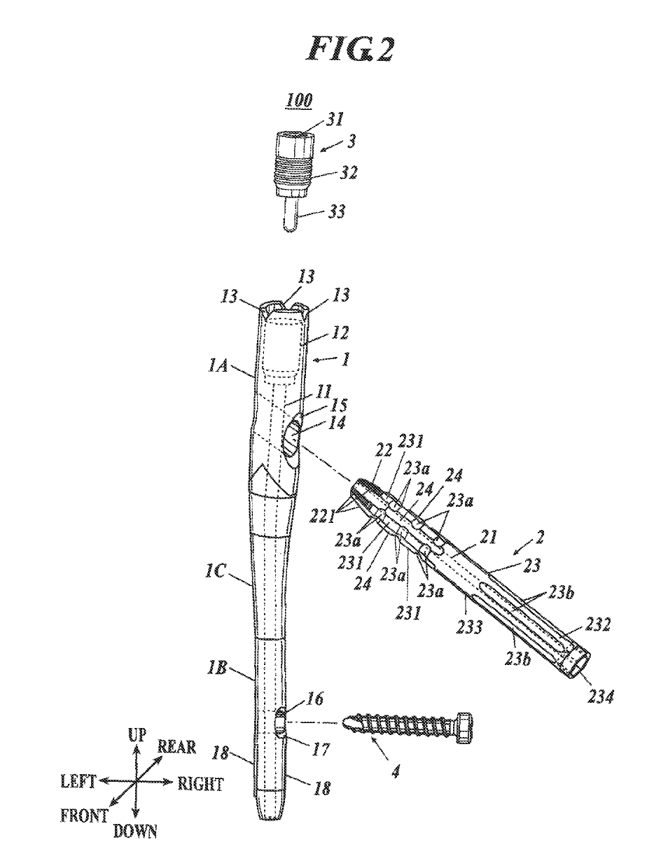

FIG. 1 is a diagram schematically showing a state of a thighbone fixing system 100 attached to a thighbone B illustrated as a preferable embodiment employing the present invention viewed from a front side and FIG. 2 is an exploded perspective view of the thighbone fixing system 100.

In the description described below, when the thighbone fixing system 100 is attached to the thighbone B, a proximal end side of the thighbone B is the upper side and a distal end side of the thighbone B is the lower side, a bone head side of the thighbone B is the left (inner) side and the opposite side of the bone head is the right (outer) side, and the stomach side is the front side and the back side is the rear side.

As shown in FIG. 1, the thighbone fixing system 100 of the present embodiment is attached and fixed to the thighbone B in order to treat a fracture of a proximal end section of the thighbone B such as a neck portion of the thighbone B.

Specifically, as shown in FIG. 1 and FIG. 2, the thighbone fixing system 100 includes an intramedullary nail main body 1 inserted into a medullary space of the thighbone body from the proximal end of a big bone piece portion (one bone piece portion) B1 of the fractured thighbone B, a bone fixing blade (bone fixing material, or component) 2 inserted through a first inserting hole 14 of the proximal end side section 1A of the intramedullary nail main body 1 and positioned on the inner side from the big bone piece portion B1 to a small bone piece portion (other bone piece portion) B2, a fixing screw 3 to fix the bone fixing blade 2 inserted through the first inserting hole 14 so that rotation is not possible and a distal screw 4 inserted through a second inserting hole 16 of a distal end side section 1B of the intramedullary nail main body 1.

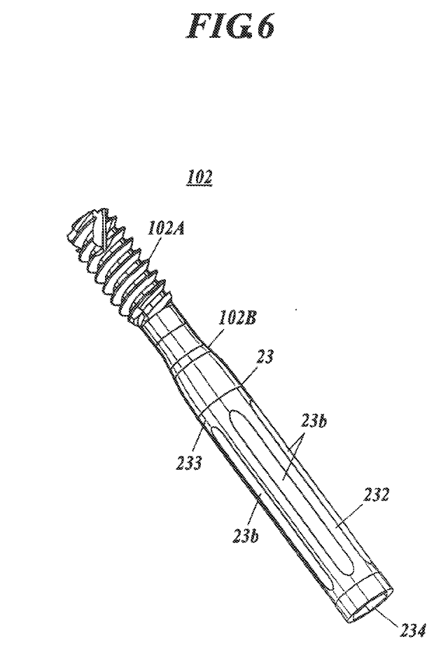

Moreover, in the thighbone fixing system 100, a bone fixing screw 102 (see FIG. 6; described later) screwed in the thighbone B can be used instead of the bone fixing blade 2 pressed in the thighbone B, depending on the state of the thighbone B of the patient.

In other words, in the thighbone fixing system 100, the bone fixing blade 2 is used for those with a relatively small cancellous bone mass such as those with weak bones or those who are aged and the bone fixing screw 102 is used for those with a relatively large cancellous bone mass such as those with strong bones or those who are young in order to fix and stabilize the thighbone B.

First, the intramedullary nail main body 1 is described in detail.

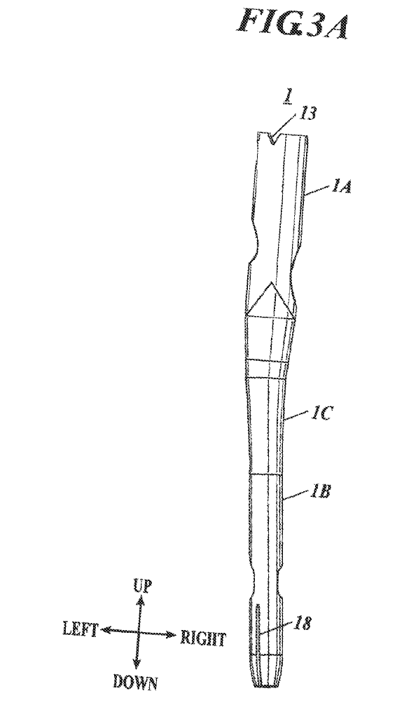

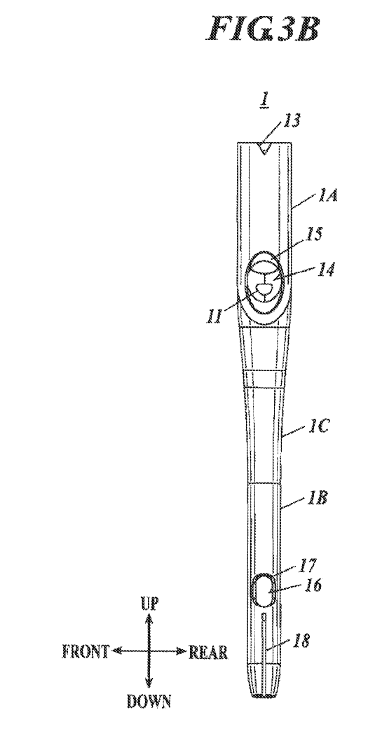

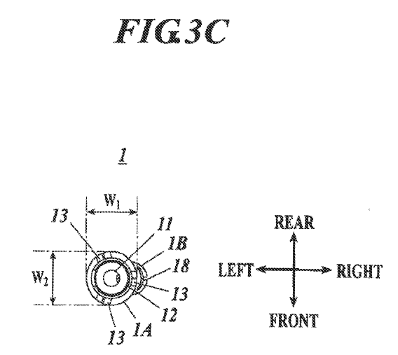

FIG. 3A is a diagram showing the intramedullary nail main body 1 viewed from the front side, FIG. 3B is a diagram showing the intramedullary nail main body 1 viewed from the right side and FIG. 3C is a diagram showing the intramedullary nail main body 1 viewed from the upper side.

As shown in FIG. 2 and FIG. 3A to FIG. 3C, the intramedullary nail main body 1 is a long stick, or shaft shape in an up and down direction when attached to the thighbone B, and is formed in a shape so that the proximal end side section 1A has a larger diameter than the distal end side section 1B and the proximal end side section 1A is bent slightly to the outer (right) side with respect to the distal end side section 1B. Moreover, in the intramedullary nail main body 1, a through hole 11 is formed in the central portion along a longitudinal direction.

In the upper portion of the proximal end side section 1A, an attachment fixing section 12 where a fixing screw 3 is attached and fixed is formed continuous with the upper end opening of the through hole 11.

Although illustration is omitted, a threaded engaged section to engage a threaded engaging section 32 (later described) of the fixing screw 3 is formed on the inner circular face of the attachment fixing section 12.

The threaded engaged section can be used to attach a target device (not shown) used in attaching and positioning of the thighbone B of the thighbone fixing system 100.

Moreover, positioning sections 13 to position the target device (not shown) are provided in an upper end edge section of the proximal end side section 1A.

A plurality (for example, three) of positioning sections 13 are provided with a predetermined space (for example, 120°) in between each other along a circular direction of the upper end edge section cut out in a substantially triangular shape from the circular upper end edge section of the proximal end side section 1A to the lower side.

Consequently, since a plurality of positioning sections 13 are provided, positioning of a target device with respect to the intramedullary nail main body 1 can be appropriately performed and attachment and positioning (later described) of the bone fixing blade 2 and the distal screw 4 with respect to the thighbone B can be accurately performed.

On the lower side of the attachment fixing section 12 of the proximal end side section 1A, the first inserting hole 14 to insert the bone fixing blade 2 through is formed. Here, the proximal end side section 1A composes an inserting hole forming section in which an inserting hole through which the bone fixing blade 2 is inserted is formed.

The inner shape of the first inserting hole 14 is formed in a substantial circular shape and is formed to penetrate through the intramedullary nail main body 1 diagonally from bottom right to top left so as to cross with the longitudinal direction of the intramedullary nail main body 1. The inner diameter of the first through hole 14 is substantially equal to or is a slightly larger diameter than the outer diameter of the bone fixing blade 2 and the bone fixing screw 102.

Moreover, for example, the first inserting hole 14 is formed in a direction to extend at about 128° with respect to the up and down direction considering an anatomical angle of the thighbone neck.

Moreover, around the right side opening of the first inserting hole 14 of the proximal end side section 1A, in other words, around the opening which is to be the inserting side of the bone fixing blade 2, a first counterbore section 15 is provided counterbored at a predetermined depth from a side face of the proximal end side section 1A to the left side (see FIG. 3B, etc.).

The first counterbore section 15 is formed so that the right side opening of the first inserting hole 14 is continuous with the side face of the proximal end side section 1A. With this, when external force is applied on the bone fixing blade 2 in a state where the bone fixing blade 2 is inserted through the first inserting hole 14, stress easily spreads to the outer side of the first inserting hole 14 and damage of the intramedullary nail main body 1 can be prevented.

Moreover, the proximal end side section 1A has a cross section shaped in a substantial elliptic shape so that a width W₂ in a front and rear direction is larger with respect to a width W₁ in a left and right direction (see FIG. 3C).

In other words, the proximal end side section 1A is formed so that the width W₂ in a direction which does not cross with the inserting direction is larger compared to the width W₁ in a direction substantially orthogonal to the longitudinal direction (up and down direction) of the intramedullary nail main body 1 and in a direction which crosses with the inserting direction of the bone fixing blade 2 through the first inserting hole 14. With this, strength of the intramedullary nail main body 1 in the proximal end side section 1A is enhanced and damage of the intramedullary nail main body 1 can be prevented when external force is applied on the proximal end side section 1A.

On the bottom end section of the proximal end side section 1A, a tapered section 1C is continuously formed with the diameter reducing toward the bottom side and a distal end side section 1B is formed continuous with the bottom end section of the tapered section 1C.

In the distal end side section 1B, a second inserting hole 16 is formed to insert the distal screw 4.

The second inserting hole 16 is formed so that the inner shape is a substantial circular shape and is formed to penetrate in the left and right direction of the distal end side section 1B.

Moreover, around the right side opening of the second inserting hole 16 of the distal end side section 1B, in other words, around the opening which is to be the inserting side of the distal screw 4, a second counterbore section 17 is provided counterbored at a predetermined depth from a side face of the distal end side section 1B to the left side (see FIG. 3B, etc.).

The second counterbore section 17 is formed so that the right side opening of the second inserting hole 16 is continuous with the side face of the distal end side section 1B. With this, when external force is applied on the distal screw 4 in a state where the distal screw 4 is inserted through the second inserting hole 16, stress easily spreads to the outer side of the second inserting hole 16 and damage of the intramedullary nail main body 1 and the distal screw 4 can be prevented.

Moreover, on a lower side than the second inserting hole 16 of the distal end side section 1B, slits 18 are provided extending along an axis direction of the intramedullary nail main body 1.

A plurality (for example, three) of slits 18 are provided with a predetermined space (for example, 120°) in between each other along a circular direction of the distal end side section 1B on the circular face section of the distal end side section 1B.

Next, the bone fixing blade 2 is described in detail.

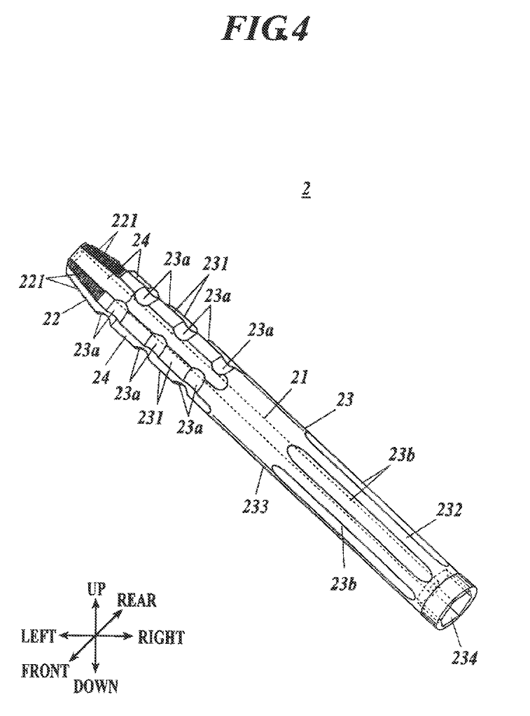

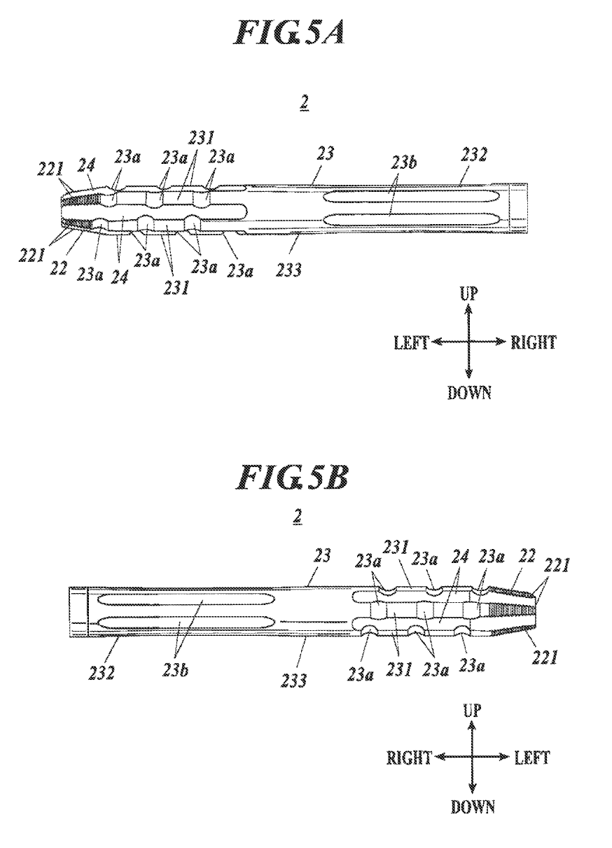

FIG. 4 is a perspective diagram showing the bone fixing blade 2. FIG. 5A is a diagram showing the bone fixing blade 2 viewed from the front side and FIG. 5B is a diagram showing the bone fixing blade 2 viewed from the rear side.

As shown in FIG. 4, FIG. 5A and FIG. 5B, the bone fixing blade 2 is a long stick shape in a predetermined direction and a through hole 21 is formed in the central portion along an axis direction. The bone fixing blade 2 includes a tip section 22 inserted first in the first inserting hole 14 and a blade main body section 23 formed continuous with the rear end of the tip section 22 (lower right end shown in FIG. 4) and has a substantially equal outer diameter along the axis direction of the bone fixing blade 2.

Groove sections 24 are formed extending along an axis direction (longitudinal direction) in a portion from a tip of the tip section 22 of the bone fixing blade 2 toward the tip section 22 of the blade main body section 23.

The groove sections 24 include a curved face in a circular shape curved toward the inner side from a surface of the tip section 22 and the blade main body section 23 and five are provided parallel with respect to each 72° with a substantially equal space in between each other along a circular direction of the bone fixing blade 2.

The tip section 22 is formed tilted at a predetermined angle with respect to the axis direction. The tip section 22 includes rib strip sections 221 where a plurality of strips of ribs protruding to the outer side from the surface are provided parallel along the longitudinal direction. Five rib strip sections 221 are provided parallel with respect to each 72° with a substantially equal space between each other along a circular direction of the bone fixing blade 2.

In other words, the diameter of the tip section 22 becomes smaller toward the tip in order to easily insert the tip section 22 in the cancellous bone of the thighbone B and by forming five groove sections 24, . . . in the circular cone trapezoidal section where a plurality of strips of circular ribs are formed on the circular face, the groove section 24 and the rib strip section 221 are in a state provided alternately in the circular direction.

The blade main body section 23 includes a plurality of cut out section forming sections or areas 231, . . . formed continuously in the rear end of each rib strip section 221.

Each of the cut out section forming sections 231 are provided with three cut out sections 23a provided along a longitudinal direction and the cut out sections 23a include a curved face in a circular shape which is cut out from the surface to the inner side along a direction crossing the axis direction of the bone fixing blade 2. Specifically, each of the cut out sections 23a are formed cut out at a predetermined length along the circular direction of the bone fixing blade 2 so as to be successively provided in a substantial right angle with respect to the groove section 24. The depth of the cut out section 23a is shallower than the depth of the groove section 24, but can be formed with substantially the same depth as the groove section 24.

A plurality (for example, five) of cut out section forming sections 231 are provided parallel with a predetermined space (for example, 72°) in between each other along a circular direction of the bone fixing blade 2. Specifically, in the blade main body section 23, five cut out section forming sections 231, . . . and five groove sections 24, . . . are provided alternately in the circular direction of the bone fixing blade 2.

In other words, as for the five cut out section forming sections 231, . . . , by forming five groove sections 24 on the blade main body section 23 in a cylinder shape, the groove section 24 and the cut out section forming section 231 are in a state provided alternately in the circular direction.

In each of the cut out section forming sections 231, cut out sections 23a adjacent to each other in the circular direction of the bone fixing blade 2 are misaligned at a predetermined amount in the axis direction and a plurality of cut out sections 23a, . . . are positioned so as to be in a spiral shape as a whole.

In other words, among the five cut out section forming sections 231, . . . , for example, with respect to any one (for example, at the most tip side) of a cut out section 23a of a first cut out section forming section 231, a cut out section 23a of a second cut out section forming section 231, which is adjacent in a clockwise direction viewed from the rear end side of the bone fixing blade 2, is provided misaligned at a predetermined amount to the rear side in the axis direction, and similarly, a cut out section 23a of a third cut out section forming section 231 adjacent in the clockwise direction with respect to the cut out section 23a of the second cut out section forming section 231 is provided misaligned at a predetermined amount to the rear side in the axis direction, and similarly, a cut out section 23a of a fourth cut out section forming section 231 adjacent in the clockwise direction with respect to the cut out section 23a of the third cut out section forming section 231 is provided misaligned at a predetermined amount to the rear side in the axis direction, and similarly, a cut out section 23a of a fifth cut out section forming section 231 adjacent in the clockwise direction with respect to the cut out section 23a of the fourth cut out section forming section 231 is provided misaligned at a predetermined amount to the rear side in the axis direction, and similarly, a cut out section 23a (for example, second from the tip) of a first cut out section forming section 231 adjacent in the clockwise direction with respect to the cut out section 23a of the fifth cut out section forming section 231 is provided misaligned at a predetermined amount to the rear side in the axis direction.

Engaged section forming sections or areas 232 are provided in the rear end side than the cut out section forming section 231 of the blade main body section 23, and five engaged sections 23b to which protruding sections 33 (later described) of the fixing screw 3 are engaged are formed on the engaged section forming sections 232. Specifically, at a rear end section of the cut out section forming section 231, a circular cylinder section 233 in which the outer diameter is substantially a circular cylinder shape is continuously formed and an engaged section forming section 232 is continuously formed in the rear end section of the circular cylinder section 233.

Each of the engaged sections 23b includes a curved face in a circular shape curved from the surface of the blade main body section 23 to the inner side, extending along the axis direction.

The five engaged sections 23b, . . . are provided parallel with respect to each 72° with a substantially equal space in between each other along the circular direction of the bone fixing blade 2. Specifically, the five engaged sections 23b, . . . are formed on the engaged section forming section 232 so as to extend in substantially the same direction as the extending direction of each of the plurality of groove sections 24, . . . .

A connecting section 234 where a bone fixing blade implanting device (not shown) of a target device (not shown) is connected is formed continuous to a rear end opening of the through hole 21 on the rear end section of the blade main body section 23.

Next, the fixing screw 3 is described.

In the fixing screw 3, a connecting section 31 to which a screw driver is connected is provided at the top end section and on the lower side of the connecting section 31, a threaded engaging section 32 to engage with the threaded engaged section formed on the inner circular face of the attachment fixing section 12 of the intramedullary nail main body 1 is formed and on the lower side of the threaded engaging section 32, a protruding section 33 to engage with the engaged section 23b of the bone fixing blade 2 is formed.

Next, the bone fixing screw 102 is described in detail.

FIG. 6 is a perspective diagram illustrating the bone fixing screw 102.

Other than the configuration described below, the bone fixing screw 102 includes a configuration substantially the same as the bone fixing blade 2, and the detailed description is omitted.

As shown in FIG. 6, a screw section 102A is formed on the bone fixing screw 102 instead of the rib strip 221, cut out section forming section 231 and groove section 24 in the bone fixing blade 2. In other words, a tapered section 102B is formed continuous with the tip side of the circular cylinder section 233 and a screw section 102A formed with a screw thread at a predetermined pitch is provided at the tip side of the tapered section 102B.

The method of providing the bone fixing screw 102 in the thighbone B is substantially the same as conventional screws. In other words, the bone fixing screw 102 is rotated in a predetermined direction to screw in the screw section 102A into the bone head and with this, the bone fixing screw 102 is provided in the thighbone B.

Each section composing the thighbone fixing system 100, in other words, the intramedullary nail main body 1, the bone fixing blade 2, the fixing screw 3, the distal screw 4 and the bone fixing screw 102 are composed of medical material such as an alloy including titanium, etc. at a predetermined ratio, stainless steel or the like.

Next, the method of attaching the thighbone fixing system 100 to the thighbone B is described.

In the present embodiment, an example of fixing the thighbone B using the bone fixing blade 2 is described.

First, the intramedullary nail main body 1, the bone fixing blade 2, the fixing screw 3 and the distal screw 4 with predetermined dimensions are selected according to an outer shape and dimensions of a fractured thighbone B and the position to implant the intramedullary nail main body 1 is specified.

Then, when the implanting position of the intramedullary nail main body 1 is specified, for example, after boring in a predetermined position of a greater trochanter of the thighbone B using a hollow awl (not shown), a guide wire (not shown) is inserted from the hole to the distal end side.

Then, the hollow awl is removed and reams a medullary cavity along the guide wire using a predetermined reamer (not shown).

After the reaming is finished, the reamer is removed, and the intramedullary nail main body 1 with the target device (not shown) positioned with the positioning section 13 and attached and fixed with the attachment fixing section 12 of the intramedullary nail main body 1 is inserted in the medullary cavity along the guide wire.

Then, an entering position of a guide pin (not shown) for pressing in the bone fixing blade 2 in the thighbone body is specified and after an entering hole of the guide pin is formed in the position, the guide pin is passed through the entering hole and the first inserting hole 14 to be entered in the thighbone B. Then, a bone fixing blade implanting hole (not shown) is formed around the guide pin using the predetermined reamer (not shown).

After forming the bone fixing blade implanting hole, the reamer is removed and a bone fixing blade implanting device (not shown) is connected to the connecting section 234 of the bone fixing blade 2 in a direction where any one of the engaged sections 23b of the bone fixing blade 2 is provided on the upper side, and the bone fixing blade 2 is pressed in the inner side of each of the big bone piece portion B1 and the small bone piece portion B2 along the guide pin. With this, the bone fixing blade 2 is positioned in the thighbone B in a state where the cut out section forming section 231 is in a position in a deeper side than the bone fracture line L.

Then, the implanting position of the distal screw 4 in the thighbone body is specified, and after forming the implanting hole in the position, the distal screw 4 is passed through the implanting hole and the second inserting hole 16 and screwed and fixed in the thighbone B.

Then, after the target device is removed from the intramedullary nail main body 1, the fixing screw 3 is attached to the attachment fixing section 12 so as to threadedly engage the threaded engaging section 32 of the fixing screw 3 to the threaded engaged section of the attachment fixing section 12. With this, the protruding section 33 of the fixing screw 3 is in a state engaged with the engaged section 23b of the bone fixing blade 2 and the bone fixing blade 2 is in a state unrotatably attached to the intramedullary nail main body 1.

As described above, according to the thighbone fixing system 100 of the present embodiment, the bone fixing blade 2 attached and fixed to the intramedullary nail main body 1 is formed in a stick shape and the bone fixing blade 2 is pressed in the inner side of each of the big bone piece portion B1 and small bone piece portion B2 so that the five cut out section forming sections 231 formed along the circular direction of the bone fixing blade 2 and provided with three cut out sections 23a cut out toward the inner side from the surface are positioned on the deeper side than the bone fracture line L. Therefore, after surgery, the cancellous bone surrounding the bone fixing blade 2 is in a state in the inner side of the cut out section 23a and even if the bone fixing blade 2 moves in the axis direction, the cut out section 23a is easily caught by the cancellous bone. Consequently, the bone fixing blade 2 can be made to not easily fall out from the thighbone B. Moreover, with the cut out section 23a, the surface area of the bone fixing blade 2 can be made larger than that of the conventional bone fixing blade 2 and the bone fixing blade 2 can be made to be supported more appropriately by pressure from the cancellous bone surrounding the bone fixing blade 2 in a state where the big bone piece portion B1 and the small bone piece portion B2 are pressed into the inner side.

Therefore, the bone fixing blade 2 falling out from the thighbone B can be prevented appropriately.

Moreover, five groove sections 24 formed along an axis direction are provided on the tip section 22 side of the bone fixing blade 2. Therefore, after surgery, the cancellous bone surrounding the bone fixing blade 2 is in a state in the inner side of the groove section 24 and even if the small bone piece portion B2 of the bone head side rotates around the bone fixing blade 2, the small bone piece portion B2 is easily caught by the groove section 24. Consequently, it can be made so that it is difficult for the bone piece portion to rotate.

Further, five units of cut out section forming sections 231 and groove sections 24 are provided respectively, and are provided alternately in the circular direction of the bone fixing blade 2. Therefore, after surgery, the cancellous bone surrounding the bone fixing blade 2 is in a state in the inner side of the cut out section 23a and the groove section 24. Consequently, the falling out from the thighbone B and the rotation of the small bone piece portion B2 can be appropriately prevented.

In addition, the cut out sections 23a provided in each cut out section forming section 231 and adjacent to each other in the circular direction are provided misaligned in the axis direction. Therefore, in removing the bone fixing blade 2 from the thighbone B after surgery, even when the cancellous bone mass surrounding the bone fixing blade 2 increases so that the pressure from the cancellous bone becomes large and it is not possible to pull out the bone fixing blade 2 simply by pulling in the axis direction, by rotating the bone fixing blade 2 in a predetermined direction (for example, counter clockwise viewed from the rear edge side) at 36°, which is half of the interval between the cut out section forming sections 231, the cancellous bone which is in the cut out section 23a before rotation is positioned on the inner side of the groove section 24 adjacent to the cut out section 23a in the counter clockwise rotating direction. With this, the cancellous bone mass on the inner side of the cut out section 23a and in a caught state can be made small and the bone fixing blade 2 can be easily pulled out in the axis direction.

In other words, according to the thighbone fixing system 100 of the present embodiment, the cancellous bone surrounding the bone fixing blade 2 is in a state in the inner side of the cut out section 23a of the bone fixing blade 2 pressed in the thighbone B and the falling out from the thighbone B can be appropriately prevented. Moreover, in removing the bone fixing blade 2 from the thighbone B after surgery, the cancellous bone in the cut out section 23a is released by simply rotating the bone fixing blade 2, and the bone fixing blade 2 can be easily pulled out in the axis direction.

In the thighbone fixing system 100, the fixing and the stabilizing of the thighbone B can be performed according to the state of the thighbone B of the patient, in other words, in those with a relatively small cancellous bone mass such as those with weak bones or those who are aged, the bone fixing blade 2 is used, whereas in those with a relatively large cancellous bone mass such as those with strong bones or those who are young, the bone fixing screw 102 is used. Consequently, a highly versatile system can be provided which can support patients with various states of the thighbone B simply by exchanging the bone fixing blade 2 with the bone fixing screw 102.

The present invention is not limited to the above described embodiments, and various modifications and changes in design can be performed without leaving the scope of the invention.

For example, the above described embodiment describes an example in which the cut out section 23a includes a curved face in a circular shape along the circular direction, however, the shape of the cut out section 23a is not limited to this.

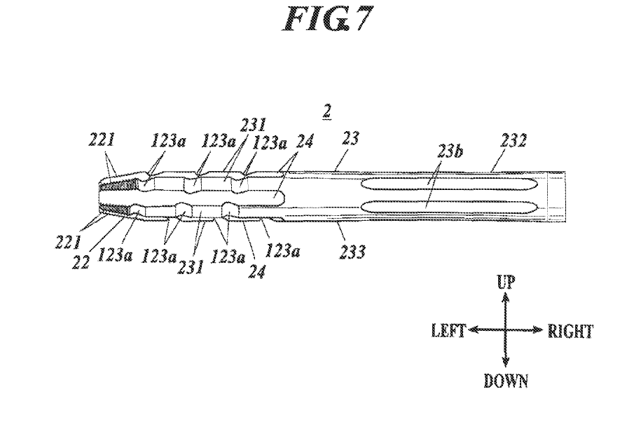

In other words, as shown in FIG. 7, a cut out section 123a including a curved face which is cut out deeper toward the tip section 22 side can be employed.

With this, an angle of a wall face of the tip section 22 side of the cut out section 123a with respect to the axis direction of the blade main body section 23 can be made larger. Therefore, the cancellous bone is in a state in the inner side of the cut out section 123a after surgery and it is easier for the cut out section 23a to be caught by the cancellous bone. Consequently, the falling out of the bone fixing blade 2 can be prevented more appropriately.

In the above described embodiment, five units of the cut out section forming sections 231 and the groove sections 24 are provided respectively in the bone fixing blade 2, however, the numbers of the cut out section forming sections 231 and the groove sections 24 are not limited to the above, and can be changed freely as necessary.

Moreover, three units of the cut out sections 23a are provided in the cut out section forming section 231, however, the number of the cut out sections 23a is not limited to this, and can be changed freely as necessary.

In addition, as for the embodiment disclosed here, it is to be regarded that all points are examples and does not limit the invention. The scope of the present invention is shown by the attached claims and not the above description, and equivalents to the claims and all changes within the scope of claims are to be included.

### INDUSTRIAL APPLICABILITY

As described above, according to the preferred embodiments of the present invention, the cancellous bone surrounding the bone fixing material is in a state in the inner side of the cut out section after surgery, and even if the bone fixing material is moved in the axis direction, the cut out section is easily caught by the cancellous bone. Moreover, with the cut out section, the surface area of the bone fixing material can be made larger, and therefore can be appropriately supported by the pressure from the cancellous bone surrounding the bone fixing material in a state pressed in the inner side of the one bone piece portion and the other bone piece portion.

As a result, the present invention can be preferably employed in a technique which can appropriately prevent a bone fixing material from falling out from a thighbone in a bone fixing material and a thighbone fixing system.

### Description of Reference Numerals

100 thighbone fixing system
1 intramedullary nail main body
1A proximal end side section
1B distal end side section
14 first inserting hole
2 bone fixing blade (bone fixing material)
231 cut out section forming section
23a cut out section
23b engaged section
24 groove section
3 fixing screw (fixing material)
33 protruding section
B thighbone
B1 big bone piece portion (one bone piece portion)
B2 small bone piece portion (other bone piece portion)

1-8. (canceled)

9. A bone fixing component attached and fixed to an intramedullary nail main body insertable in a medullary cavity of a fractured thighbone to fix a first bone piece portion and a second bone piece portion different from the first bone piece portion composing the thighbone, the bone fixing component comprising:
a form formed in a shaft shape and having a longitudinal direction; and
at least one a cut out section area provided with at least one cut out section, the cut out section formed along a direction which crosses with the longitudinal direction of the bone fixing component and cut out from an outer surface toward an inner side, wherein
the bone fixing component is insertable into an inner side of each of the first bone piece portion and the second bone piece portion so that the cut out section area is inserted first so that it extends deeper into the bone portions than the remainder of the bone fixing component.

10. The bone fixing component according to claim 9, further comprising at least one groove section cut out along the longitudinal direction on a tip section, the tip section insertable first into the first bone piece portion and the second bone piece portion, the groove section formed curved inwardly toward the inner side from the outer surface.

11. The bone fixing component according to claim 10, wherein the at least one cut out section area and the at least one groove section are each provided in a plurality of units and are provided alternately in a circular direction substantially orthogonal to the longitudinal direction of the bone fixing component.

12. The bone fixing component of claim 9, wherein
the cut out section area is provided in a plurality of units with a predetermined space in between each unit in the circular direction substantially orthogonal to the longitudinal direction; and
the cut out sections provided in each of the plurality of cut out section areas are adjacent to each other in the circular direction and are provided misaligned in the longitudinal direction.

13. The bone fixing component of claim 10, wherein
the cut out section area is provided in a plurality of units with a predetermined space in between each unit in the circular direction substantially orthogonal to the longitudinal direction; and
the cut out sections provided in each of the plurality of cut out section areas are adjacent to each other in the circular direction and are provided misaligned in the longitudinal direction.

14. The bone fixing component of claim 11, wherein
the cut out section area is provided in a plurality of units with a predetermined space in between each unit in the circular direction substantially orthogonal to the longitudinal direction; and
the cut out sections provided in each of the plurality of cut out section areas are adjacent to each other in the circular direction and are provided misaligned in the longitudinal direction.

15. The bone fixing component of claim 9, further comprising a tip section, the tip section insertable first into the first bone portion and the second bone portion, wherein the at least one cut out section is cut out deeper toward the tip section.

16. The bone fixing component of claim 10, further comprising a tip section, the tip section insertable first into the first bone portion and the second bone portion, wherein the at least one cut out section is cut out deeper toward the tip section.

17. The bone fixing component of claim 11, further comprising a tip section, the tip section insertable first into the first bone portion and the second bone portion, wherein the at least one cut out section is cut out deeper toward the tip section.

18. The bone fixing component of claim 12, further comprising a tip section, the tip section insertable first into the first bone portion and the second bone portion, wherein the at least one cut out section is cut out deeper toward the tip section.

19. The bone fixing component of claim 13, further comprising a tip section, the tip section insertable first into the first bone portion and the second bone portion, wherein the at least one cut out section is cut out deeper toward the tip section.

20. The bone fixing component of claim 14, further comprising a tip section, the tip section insertable first into the first bone portion and the second bone portion, wherein the at least one cut out section is cut out deeper toward the tip section.

21. A thighbone fixing system comprising:
a bone fixing component comprising:
a form formed in a shaft shape; and
a cut out section area provided with at least one cut out section formed along a direction which crosses with a longitudinal direction of the bone fixing component and cut out from an outer surface toward an inner side, wherein the bone fixing component is insertable into an inner side of each of a first bone piece portion and a second bone piece portion so that the cut out section area is inserted first so that it extends deeper into the bone than the remainder of the bone fixing component; and
an intramedullary nail main body formed in a shaft shape and having an intramedullary nail main body longitudinal direction, wherein the intramedullary nail main body includes a proximal end section and a distal end section, an insertion hole formed to extend across the proximal end section in an insertion direction, the insertion hole shaped to receive the bone fixing component, wherein a cross section of the proximal end section taken orthogonal to the intramedullary nail main body longitudinal direction is substantially elliptical, the cross section having a first width extending across the proximal end section and orthogonal to the insertion direction and a second width extending across the proximal end section and aligned with the insertion direction, wherein the first width is greater than the second width.

22. The thighbone fixing system according to claim 21, further comprising at least one groove section cut out along the bone fixing component longitudinal direction on a tip section insertable first into the first bone piece portion and the second bone piece portion, the groove section formed curved inwardly toward the inner side from the surface.

23. The thighbone fixing system according to claim 22, wherein the at least one cut out section area and the at least one groove section are each provided in a plurality of units and are provided alternately in a circular direction substantially orthogonal to the longitudinal direction of the bone fixing component.

24. The thighbone fixing system according to claim 21, wherein
the cut out section area is provided in a plurality of units with a predetermined space in between each unit in the circular direction substantially orthogonal to the longitudinal direction of the bone fixing component; and
the cut out sections provided in each of the plurality of cut out section areas are adjacent to each other in the circular direction and are provided misaligned in the longitudinal direction of the bone fixing component.

25. The thighbone fixing system according to claim 22, wherein
the cut out section area is provided in a plurality of units with a predetermined space in between each unit in the circular direction substantially orthogonal to the longitudinal direction of the bone fixing component; and
the cut out sections provided in each of the plurality of cut out section areas are adjacent to each other in the circular direction and are provided misaligned in the longitudinal direction of the bone fixing component.

26. The thighbone fixing system according to claim 23, wherein
the cut out section area is provided in a plurality of units with a predetermined space in between each unit in the circular direction substantially orthogonal to the longitudinal direction of the bone fixing component; and
the cut out sections provided in each of the plurality of cut out section areas are adjacent to each other in the circular direction are and provided misaligned in the longitudinal direction of the bone fixing component.

27. The thighbone fixing system according to claim 21, further comprising a tip section, the tip section insertable first into the first bone portion and the second bone portion, wherein the at least one cut out section is cut out deeper toward the tip section side.

28. The thighbone fixing system according to claim 22, further comprising a tip section, the tip section insertable first into the first bone portion and the second bone portion, wherein the at least one cut out section is cut out deeper toward the tip section.

29. The thighbone fixing system according to claim 23, further comprising a tip section, the tip section insertable first into the first bone portion and the second bone portion, wherein the at least one cut out section is cut out deeper toward the tip section.

30. The thighbone fixing system according to claim 24, further comprising a tip section, the tip section insertable first into the first bone portion and the second bone portion, wherein the at least one cut out section is cut out deeper toward the tip section.

31. The thighbone fixing system according to claim 25, further comprising a tip section, the tip section insertable first into the first bone portion and the second bone portion, wherein the at least one cut out section is cut out deeper toward the tip section.

32. The thighbone fixing system according to claim 26, further comprising a tip section, the tip section insertable first into the first bone portion and the second bone portion, wherein the at least one cut out section is cut out deeper toward the tip section.
